(19) United States
(12) Patent Application Publication
(10) Pub. No.: US 2025/0213379 A1
(43) Pub. Date: Jul. 3, 2025

(54) ASSIST DEVICE FOR KNEE JOINT

(71) Applicant: NATIONAL TAICHUNG UNIVERSITY OF SCIENCE AND TECHNOLOGY, Taichung (TW)

(72) Inventors: Bor-Tsang Wu, Taichung (TW); Mei-Ying Kuo, Taichung (TW); Chi-Chung Kuo, Taichung (TW); Yi-Ru Jheng, Kaohsiung (TW); Yi-Zhen Li, Changhua County (TW); Yi-Ting Hsiao, Changhua County (TW); Ying-Jhen Lu, Taichung (TW); Yi-Chen Wu, Hsinchu (TW)

(21) Appl. No.: 18/986,140

(22) Filed: Dec. 18, 2024

(30) Foreign Application Priority Data
Dec. 28, 2023 (TW) 112151238

(51) Int. Cl. A61F 5/01

(52) U.S. Cl. CPC A61F 5/0123

(57) ABSTRACT

An assist device for a knee joint is provided. The assist device includes a knee joint assist mechanism including a first bracket connected to a waist fixation mechanism and fixed to the lower limb and arranged along the user's thigh, a first spacing adjustment member pivotally connected to one end of the first bracket, a second bracket arranged along the user's calf, and a second spacing adjustment member pivotally connected to one end of the second bracket, in which the first spacing adjustment member and the second spacing adjustment member are engaged with each other, and respectively have coupling structures with different sizes. When the knee joint is in an extended state, the first spacing adjustment member and the second spacing adjustment member have a larger center distance formed therebetween, such that support is provided to the user's knee joint to reduce wear and defect thereof.

### CROSS-REFERENCE TO RELATED APPLICATION

The present application claims priority to and the benefit of Taiwanese Patent Application No. 112151238 under 35 U.S.C. § 119, filed on Dec. 28, 2023, in the Taiwan Intellectual Property Office, the entire disclosure of which is incorporated herein by reference.

### BACKGROUND OF THE INVENTION

### 1. Field of the Invention

The present invention relates to an assist device for a knee joint, and more particularly to an assist device for a knee joint having spacing adjustment members with coupling structures of different sizes arranged at a position corresponding to the knee joint, such as two ratchets with teeth of different tooth heights, such that when the user's knee joint is in an extended state, the coupling structures of the two spacing adjustment members are engaged with one another to have a large distance, thereby providing support to the user's knee joint and reducing wear and defect for the user's knee joint.

### 2. Description of the Related Art

Osteoarthritis (OA) is a chronic progressive joint disease that commonly affects the knee joint, which primarily impacts the cartilage tissue of joints such as the knee, hip, finger joints, and spine. The primary causes of osteoarthritis include the degeneration of joint function with age, frequent flexion and extension movements of the legs leading to joint wear, and increased pressure on the joints due to obesity. The primary characteristic of osteoarthritis is cartilage wear. Cartilage is a soft tissue that covers the joints, helping to reduce friction between bones and facilitate smooth joint movement. In osteoarthritis, the joint cartilage gradually wears away, leading to direct bone-on-bone friction, which causes pain, swelling, and limited joint function. This condition can further result in swelling of the surrounding soft tissues, exacerbating cartilage wear.

To alleviate the pain and cartilage wear experienced by osteoarthritis patients, various assistive devices have been developed. These devices aim to provide more convenient, flexible, and multifunctional support for osteoarthritis patients, enhancing their mobility while reducing pain, physical strain, and cartilage wear. For example, a cane is one of the common assistive devices, used to provide support for users by holding it in their hands. Additionally, exoskeleton devices are also common assistive tools that can be attached to the patient's legs to aid in walking and provide support, thereby reducing the pressure during standing or walking. However, conventional exoskeleton devices are typically equipped with power components such as motors and power supplies, making their structure quite complex, which results in a conspicuous appearance and makes them cumbersome and inconvenient to use.

Therefore, designing a knee joint assistive device to address the shortcomings of conventional technology and enhance its practical application in the industry is a significant topic for the inventors of this invention.

### SUMMARY OF THE INVENTION

In view of the above-referenced technical inadequacies, the inventors, leveraging years of research and practical experience in the relevant field, have developed an assist device for the knee.

In order to solve the above-mentioned problems existing in the art, the present invention provides at least one knee joint auxiliary mechanism, which is arranged at least on a side of a lower limb of a user, and the knee joint assist mechanism includes a first bracket, a first spacing adjustment member, a second spacing adjustment member, and a second bracket. One end of the first bracket is connected to a waist fixation mechanism and fixed to the lower limb through a waist fixation belt of the waist fixation mechanism. The first spacing adjustment member is pivotally connected to the other end of the first bracket corresponding to a position of a knee joint of the lower limb, and the second spacing adjustment member is movably engaged with the first spacing adjustment member. One end of the second bracket is pivotally connected to the second spacing adjustment member at a position corresponding to the knee joint, and the other end of the second bracket contacts the ground. Each of the first spacing adjustment member and the second spacing adjustment member has a plurality of base coupling structures and a plurality of support coupling structures having sizes that are larger than sizes of the plurality of base coupling structures. When the knee joint is in a bent state, the base coupling structures of the first spacing adjustment member and the base coupling structures of the second distance adjusting member are engaged with one another, and a first relative distance is formed between the first distance adjusting member and the second distance adjusting member. When the knee joint is in an extended state, the support coupling structures of the first spacing adjustment member and the support coupling structures of the second spacing adjustment member are engaged with one another, a second relative distance is formed between the first spacing adjustment member and the second spacing adjustment member, and the second relative distance is greater than the first relative distance.

Preferably, the waist fixation mechanism includes the waist fixation mechanism mounted on a waist and a hip support having one end connected to the waist fixation belt and supporting a hip. The waist fixation belt is sleeved onto a lower side of the user's anterior superior iliac spine. The hip support is a plate-shaped structure corresponding to a shape of the hip, and one end of the hip support is detachably connected to the waist fixation belt through a hip support fixing member.

Preferably, the waist fixing mechanism is worn on the lower limb and pants-shaped, and the waist fixation mechanism includes the waist fixation belt sleeved onto the waist, two thigh fixation belts respectively sleeved onto two upper thighs, and two thigh lifting belts connected between the waist fixation belt and the two thigh fixation belts. The first bracket of the at least one knee joint auxiliary mechanism is fixed to the lower limb through the waist fixation mechanism.

Preferably, a difference between the second relative distance and the first relative distance gradually increases by one or more intermediate coupling structures between the base coupling structures and the one or more support coupling structures, with the heights of the one or more intermediate coupling structures gradually increase from a direction from the base coupling structures to the one or more support coupling structures.

Preferably, a maximum value of the difference between the second relative distance and the first relative distance corresponds to a maximum pain point of the user.

Preferably, the assist device includes two of the knee joint auxiliary mechanism, and each of the two knee joint assist mechanisms includes the first bracket, the first spacing adjustment member, the second spacing adjustment member and the second bracket that are arranged on an outer side of the lower limb, and each of the first bracket arranged on the outer side of the lower limb is fixed to the lower limb through the waist fixation mechanism.

Preferably, each of the two knee joint assist mechanisms includes another first bracket, another first spacing adjustment member, another second spacing adjustment member, and another second bracket that are arranged on an inner side of the lower limb, and the first brackets respectively arranged on the outer side and the inner side of the lower limb are both fixed to the lower limb through the waist fixation mechanism.

Preferably, the first spacing adjustment member is a ratchet, and the second spacing adjustment member is a ratchet or a ratchet base. The plurality of base coupling structures and the plurality of support coupling structures of the first spacing adjustment member are ratchet teeth, and the second spacing adjustment member are provided with ratchet teeth or tooth grooves corresponding to the plurality of base coupling structures and the one or more support coupling structures of the first spacing adjustment member.

Preferably, the knee joint auxiliary mechanism includes at least one leg fixation member in a ring shape surrounding the user's leg.

Preferably, the knee joint assist mechanism includes at least one foot fixation member, which is arranged at the other end of the second bracket and is detachably connected to the second bracket and a side surface of the user's shoe to fix the other end of the second bracket to the user's foot.

In conclusion, the assist device for the knee joint provided by the present invention features a simple structure and is easy to operate. The assist device does not include power components such as motors or power supplies. Instead, the assist device responds to the user's movements to rotate the first spacing adjustment member and the second spacing adjustment member, thereby altering the overall height of the assist device for the knee joint. Both the first spacing adjustment member and the second spacing adjustment member are equipped with multiple base coupling structures and multiple support coupling structures that are larger than the base coupling structures. For example, the first spacing adjustment member and the second spacing adjustment member can be ratchets with teeth of different sizes. As the user transitions from a sitting position to a standing position, the knee joint moves from a bent state to an extended state, causing the first spacing adjustment member and the second spacing adjustment member to rotate simultaneously, which allows the first spacing adjustment member and the second spacing adjustment member to smoothly transition from a state where the base coupling structures are engaged with one another to a state where the support coupling structures are engaged with one another. Consequently, a center distance between the first spacing adjustment member and the second spacing adjustment member increases, distributing the pressure on the knee joint through the first bracket and the second bracket of the knee joint auxiliary mechanism, as well as the waist fixation mechanism, to the waist, legs, and feet, thereby providing effective knee joint support. The assist device for the knee joint of the present invention can adjust its overall height in response to the user's posture changes. It does not require external power, providing support to the knee joint during posture transitions, so as to effectively prevent direct friction between the knee joints, reducing pain and cartilage wear.

Additionally, the waist fixation mechanism of the assist device for the knee joint provided by the present invention includes a waist fixation belt and a hip support. One end of the hip support is detachably connected to the waist fixation belt through a hip support fixing member, so as to allow users to easily detach or adjust the position of the hip support by unfastening the hip support fixing member, facilitating restroom use without needing to remove the entire assist device for the knee joint. Moreover, the first bracket, the first spacing adjustment member, the second spacing adjustment member, the second bracket, the foot fixation member, and the leg fixation member of each of the knee joint auxiliary mechanism of the assist device for the knee joint of the present invention can be concealed under the user's clothing and shoes, thereby maintaining an aesthetically pleasing appearance while providing effective support to the knee joint.

### BRIEF DESCRIPTION OF THE DRAWINGS

In order to more clearly illustrate the technical solution of the present invention, the following will briefly introduce the drawings required for use in the embodiments;

FIG. 1 is a schematic diagram of an assist device for a knee joint according to a first embodiment of the present invention;

FIG. 2a is a schematic diagram of a first spacing adjustment member and a second spacing adjustment member of the assist device when the user's knee joint is in a bent state according to the first embodiment of the present invention;

FIG. 2b is a schematic diagram of the first spacing adjustment member and the second spacing adjustment member of the assist device when the user's knee joint is in an extended state according to the first embodiment of the present invention;

FIG. 3 is a schematic diagram of an assist device for a knee joint according to a second embodiment of the present invention;

FIG. 4 is a schematic diagram of an assist device for a knee joint according to a third embodiment of the present invention;

FIG. 5a is a schematic diagram of a first spacing adjustment member and a second spacing adjustment member of the assist device when the user's knee joint is in a bent state according to the second embodiment of the present invention;

FIG. 5b is a schematic diagram of the first spacing adjustment member and the second spacing adjustment member of the assist device when the user's knee joint is in an extended state according to the second embodiment of the present invention;

FIG. 6a is a schematic diagram of a first spacing adjustment member and a second spacing adjustment member of the assist device when the user's knee joint is in a bent state according to the third embodiment of the present invention; and

FIG. 6b is a schematic diagram of the first spacing adjustment member and the second spacing adjustment member of the assist device when the user's knee joint is in an extended state according to the third embodiment of the present invention.

### DETAILED DESCRIPTION OF THE PREFERRED EMBODIMENTS

The advantages, features and technical methods achieved by the present invention will be described in more detail with reference to exemplary embodiments and the attached drawings so as to be easier to understand. The present invention can be implemented in different forms and should not be understood to be limited to the embodiments described herein. On the contrary, for those with ordinary knowledge in the relevant technical field, the provided embodiments will make the present disclosure more thorough and comprehensive and fully convey the scope of the present invention, and the scope of protection of the present invention will be defined only by the scope of the attached patent application.

It should be understood that although the terms “first”, “second”, etc. may be used in the present invention to describe various elements, components, regions, layers and/or parts, these elements, components, regions, layers and/or parts should not be limited by these terms. These terms are only used to distinguish one element, component, region, layer and/or section from another element, component, region, layer and/or section. Therefore, the “first element”, “first component”, “first region”, “first layer” and/or “first portion” discussed below may be referred to as “second element”, “second component”, “second region”, “second layer” and/or “second portion” without departing from the spirit and teachings of the present invention.

In addition, the terms “include” and/or “comprising” refer to the existence of the stated features, regions, wholes, steps, operations, elements and/or parts, but do not exclude the existence or addition of one or more other features, regions, wholes, steps, operations, elements, parts and/or combinations thereof.

Unless otherwise defined, all terms (including technical and scientific terms) used in the present invention have the same meaning as commonly understood by one of ordinary skill in the art to which the present invention belongs. It will be further understood that those terms as defined in commonly used dictionaries should be interpreted as having definitions that are consistent with their meanings in the context of the relevant art and the present invention, and will not be interpreted as idealized or overly formal meanings unless expressly so defined herein.

The present invention will be further described in detail below with reference to the accompanying drawings. These drawings are simplified schematic diagrams, which only illustrate the basic structure of the present invention in a schematic manner, and the proportions and sizes of the components are exaggerated for the sake of clarity. Therefore, these drawings are not intended to limit the present invention.

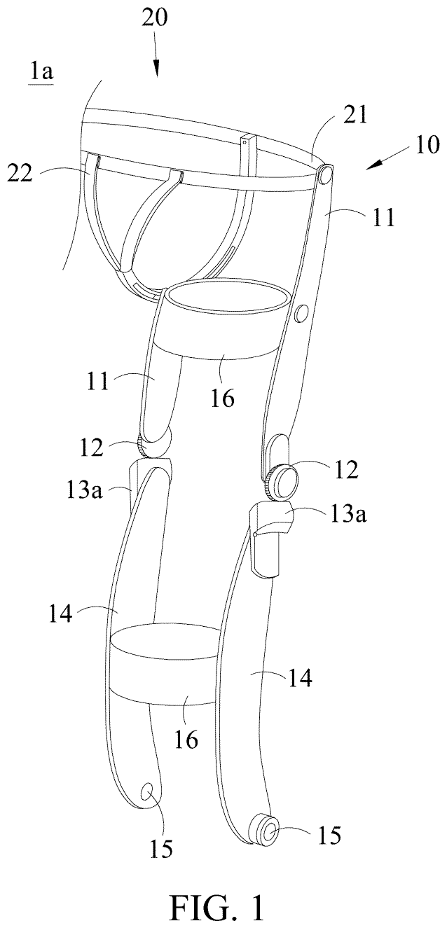

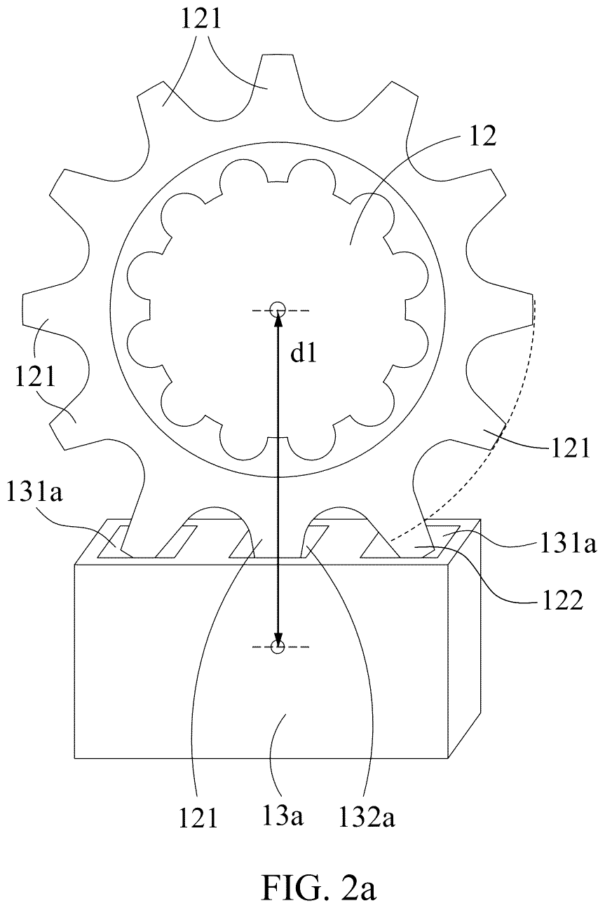

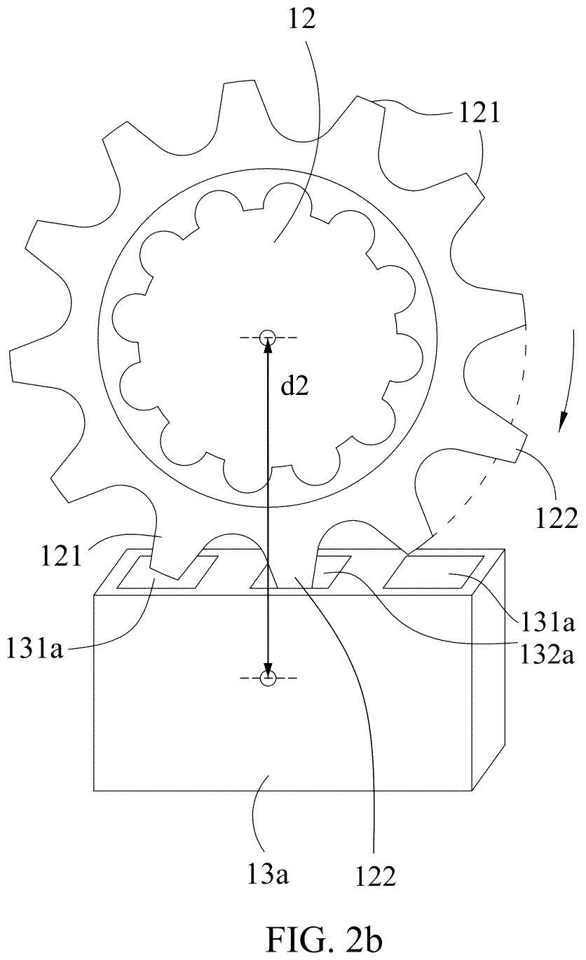

Referring to FIGS. 1, 2a and 2b, FIG. 1 is a schematic diagram of an assist device for an knee joint according to a first embodiment of the present invention; FIG. 2a is a schematic diagram of a first spacing adjustment member and a second spacing adjustment member of the assist device when the user's knee joint is in a bent state according to the first embodiment of the present invention; and FIG. 2b is a schematic diagram of the first spacing adjustment member and the second spacing adjustment member of the assist device when the user's knee joint is in an extended state according to the first embodiment of the present invention.

As shown in FIG. 1, according to the first embodiment of the present invention, an assist device 1a for the knee joint is provided, and the assist device 1a includes at least one knee joint auxiliary mechanism 10, which is arranged at least on a side of a lower limb of a user, and the knee joint assist mechanism each includes a first bracket 11, a first spacing adjustment member 12, a second spacing adjustment member 13a, and a second bracket 14. One end of the first bracket 11 is connected to a waist fixation mechanism 20 and fixed to the lower limb through a waist fixation belt 21 of the waist fixation mechanism 20. The first spacing adjustment member 12 is pivotally connected to another end of the first bracket 11 corresponding to a position of the knee joint of the lower limb, and the second spacing adjustment member 13a is movably engaged with the first spacing adjustment member 12. One end of the second bracket 14 is pivotally connected to the second spacing adjustment member 13a at a position corresponding to the knee joint, and another end of the second bracket 14 contacts the ground. In addition, the waist fixation belt 21 may be provided with a hip support 22, in which one end of the hip support 22 is rotatably connected to the waist fixation belt 21, and another end or another side of the hip support 22 is detachably buckled to the waist fixation belt 21; thereby, it is convenient for the user to put on and take off the assist device 1a of the present invention.

The first spacing adjustment member 12 may be provided with first base coupling structures 121 and one or more first support coupling structures 122 having sizes (e.g., heights) that are larger than sizes of the first base coupling structures 121, and the second spacing adjustment member 13a may be provided with side coupling structures 131a and one or more center coupling structures 132a having sizes (e.g., depths) that are larger than sizes of the side coupling structures 131a. Nevertheless, the present disclosure is not limited thereto, the center coupling structure 132a may have the same size as the side coupling structures 131a. When the knee joint is in a bent state, the first base coupling structures 121 of the first spacing adjustment member 12 may be coupled to (e.g., be received in) the center coupling structure 132a of the second spacing adjustment member 13a, and a first relative distance d1 may be formed between the first spacing adjustment member 12 and the second spacing adjustment member 13a. At this state, the first support coupling structures 122 may or may not couple to (e.g., be received in or be partially received in) the side coupling structures 131a. When the knee joint is in an extended state, the first support coupling structure 122 of the first spacing adjustment member 12 may be coupled to (e.g., be received in) the center coupling structure 132a of the second spacing adjustment member 13a, a second relative distance d2 may be formed between the first spacing adjustment member 12 and the second spacing adjustment member 13a. Since the first support coupling structure 122 is provided with a larger size than the first base coupling structures 121, while being received in the center coupling structure 132a, the second relative distance d2 may be greater than the first relative distance d1.

Specifically, as shown in FIG. 2a, in this embodiment, the first spacing adjustment member 12 may be a cam or a ratchet, preferably a ratchet, and the second spacing adjustment member 13a may be a supporting plate corresponding to the cam or a ratchet base corresponding to the ratchet, preferably the ratchet base. The first spacing adjustment member 12 may be provided with base ratchet teeth as the first base coupling structures 121 and one or more supporting ratchet teeth as the first support coupling structures 122, and the second spacing adjustment member 13a has side tooth grooves as the side coupling structures 131a and one or more center tooth grooves as the center coupling structures 132a. In this embodiment, the supporting ratchet teeth may be provided with a tooth height higher than that of the base ratchet teeth, and the side tooth grooves and the center tooth grooves may have the same tooth groove depth.

Furthermore, as shown in FIG. 2a, when the knee joint is in a bent state, the base ratchet teeth of the first spacing adjustment member 12 and the center tooth groove of the second spacing adjustment member 13a may be coupled with each other, and the first relative distance d1 is formed between the first spacing adjustment member 12 and the second spacing adjustment member 13a. At this state, the supporting ratchet tooth may or may not be received in or mesh with the side tooth grooves. Moreover, as shown in FIG. 2b, when the knee joint is in the extended state, the supporting ratchet teeth of the first spacing adjustment member 12 and the center tooth groove of the second spacing adjustment member 13a may be coupled with each other, the second relative distance d2 is formed between the first spacing adjustment member 12 and the second spacing adjustment member 13a., And since the tooth height of the supporting ratchet teeth (i.e., the first support coupling structures 122) is greater than the tooth height of the base ratchet teeth (i.e., the first base coupling structures 121), the second relative distance d2 is greater than the first relative distance d1.

Consequently, users suffering from knee joint illness such as osteoarthritis may wear the assist device 1a of the present invention to provide support for their knee joints. When the user is in a seated position with the knee joint bent, the first base coupling structures 121of the first spacing adjustment member 12 and the center coupling structures 132a of the second spacing adjustment member 13a may engage with each other, and the first relative distance d1 is formed between the first spacing adjustment member 12 and the second spacing adjustment member 13a. As the user transitions from the seated position with the knee joint bent to a standing position with the knee joint extended, the user's thigh moves the first bracket 11, causing the first spacing adjustment member 12 and the second spacing adjustment member 13a to pivot and change their engagement point. The first bracket 11 rotates the first spacing adjustment member 12 along a rotational axis, and the second spacing adjustment member 13a, which is engaged with the first spacing adjustment member 12, follows this movement. This causes the first spacing adjustment member 12 and the second spacing adjustment member 13a to rotate, such that first support coupling structures 122 is engaged with the center coupling structure 132a. Consequently, the second relative distance d2 larger than the first relative distance d1 may be formed between the first spacing adjustment member 12 and the second spacing adjustment member 13a.

With this configuration, as the user's knee joint transitions from the bent state to the extended state, the engagement between the first spacing adjustment member 12 and the second spacing adjustment member 13a changes in response to the user's movements without the need for additional power. This causes the first support coupling structures 122 of the first spacing adjustment member 12 and the center coupling structure 132a of the second spacing adjustment member 13a to engage with one another. Consequently, the overall height of the knee joint auxiliary mechanism 10 may be adjusted through switching between the first base coupling structures 121 and the first support coupling structures 122, and providing support to reduce the pressure on the knee joint, prevent direct friction between the knee joints, and alleviate pain, swelling, and cartilage wear.

In this embodiment, a difference between the first relative distance d1 and the second relative distance d2 may range from 0.3 cm to 3 cm, and ratchets with different tooth heights may be selected as the first spacing adjustment member 12 according to actual needs, such that the difference between the first relative distance d1 and the second relative distance d2 may be adjusted within an appropriate range. Furthermore, given that the first spacing adjustment member 12 is provided with multiple first base coupling structures 121 and one or more first support coupling structures 122, any appropriately sized or type of ratchet may be used as the first spacing adjustment member 12. For example, ratchets with suitable module, diameter, number of teeth, tooth width, tooth height, material, may be selected as the first spacing adjustment member 12. Alternatively, other suitable types of components can be used as the first spacing adjustment member 12, and the present invention is not limited thereto.

Additionally, in this embodiment, the assist device 1a of this invention includes two knee joint auxiliary mechanisms 10, each including two first brackets 11, two first spacing adjustment members 12, two second spacing adjustment members 13a, and two second brackets 14. The two first brackets 11, the two first spacing adjustment members 12, the two second spacing adjustment members 13a, and the two second brackets 14 of each of the knee joint auxiliary mechanisms 10 are positioned on inner and outer sides of one of the user's lower limbs. Furthermore, in this embodiment, the assist device 1a of the present invention includes the waist fixation mechanism 20, which includes the waist fixation mechanism 21 mounted on the waist and a hip support 22 with two ends connected to the waist fixation belt 21 and supporting the hip. It is worth mentioning that the waist fixation belt 21 is sleeved onto a lower side of the user's anterior superior iliac spine. Additionally, the hip support 22 is designed in a plate-like structure that conforms to the shape of the hip, and generally forms a Y-shape from the front to the back of the user, providing ample support to the hips while avoiding the user's private areas. For instance, a straight part of the hip support 22 may fit into the user's gluteal groove, effectively helping to transfer the pressure or force experienced by the user during the transition of the knee joint from the bent state to the extended state to the assist device for the knee joint of this invention, which also enhances the comfort of the user when wearing the assist device 1a. Furthermore, one end of the hip support 22 is detachably connected to the rear side of the waist fixation belt 21 via a buckle fastener. With the configuration of the hip support 22 and the buckle fastener, the user may easily unfasten the buckle fastener to remove or adjust the position of the hip support 22 when using the restroom, which allows the user to conveniently use the restroom without needing to remove the entire assist device 1a.

Furthermore, the first brackets 11 of the two knee joint auxiliary mechanisms 10 are respectively connected to the waist fixation belt 21 and the hip support 22 of the waist fixation mechanism 20. Specifically, in one of the knee joint auxiliary mechanisms 10, one end of the first bracket 11 is pivotally connected to a side of the waist fixation belt 21, another end of the first bracket 11 corresponding to the position of the knee joint is pivotally connected to the first spacing adjustment member 12, one end of the second bracket 14 corresponding to the position of the knee joint is pivotally connected to the second distance adjusting member 13a, and the first spacing adjustment member 12 and the second spacing adjustment member 13a are engaged with each other. Furthermore, in the same knee joint auxiliary mechanism 10, one end of another first bracket 11 is pivotally connected to the lower side of the hip support body 22, and another end of another first bracket 11 corresponding to the position of the knee joint is pivotally connected to the first spacing adjustment member 12. One end of the second bracket 14 corresponding to the position of the knee joint is pivotally connected to the second spacing adjustment member 13a, and the first spacing adjustment member 12 and the second spacing adjustment member 13a are engaged with each other.

For the sake of simplicity in the illustration, only the knee joint auxiliary mechanism 10 on the user's left lower limb is shown in FIG. 1, while the knee joint auxiliary mechanism 10 on the user's right lower limb is omitted. It is understood by those skilled in the art that the knee joint auxiliary mechanism 10 on the user's left lower limb can have a structure similar to that shown in FIG. 1. Therefore, further elaboration is omitted.

With this configuration, the two knee joint auxiliary mechanisms 10 of the assist device 1a may provide support from both the outer and inner sides of the leg. Additionally, the waist fixation mechanism 20 supports the user's anterior superior iliac spine and perineum, effectively distributing body weight, thereby achieving more stable and even knee joint assistance, preventing potential issues such as body misalignment, shifting, and deformation that could occur with support structures only on the outer side.

In addition, the two knee joint auxiliary mechanisms 10 of the assist device 1a of the present invention each include at least one leg fixation member 16 in a ring shape surrounding the user's leg. Specifically, the knee joint auxiliary mechanism 10 includes two leg fixation members 16 respectively surrounding the user's thigh and calf.

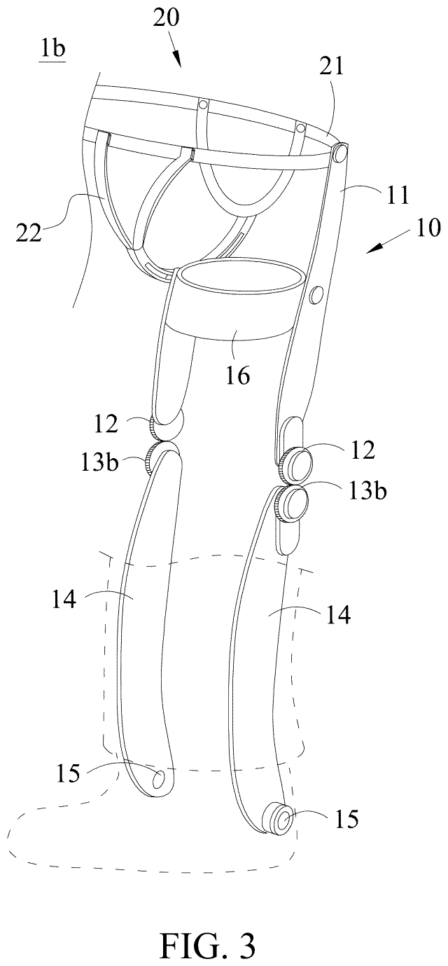

Furthermore, each of the two knee joint assist mechanisms 10 of the assist device 1a of the present invention includes at least one foot fixation member 15, which is arranged at another end of the second bracket 14 and is detachably connected to the second bracket 14 and a side surface of the user's shoe to fix another end of the second bracket 14 to the user's foot. Specifically, the knee joint auxiliary mechanism 10 includes two foot fixation members 15 respectively disposed at movable ends of the two second brackets 14, which may be a combination of a rivet and a rivet base. The two second brackets 14 may be inserted into the user's shoes through the two foot fixation members 15, and respectively fixed to the two side surfaces of the user's shoes through the foot fixing members 15. In addition, the first bracket 11, the first spacing adjustment member 12, the second spacing adjustment member 13a and the second brackets 14 of the knee joint assist mechanism 10 may also be worn on the inside of the user's clothing, for example, covered by the trouser legs (as shown by the dotted line in FIG. 3), such that the assist device 1a of the present invention can be concealed, providing support to the knee joint while maintaining aesthetically pleasing appearance.

In addition, in this embodiment, the first spacing adjustment member 12 may have eleven first base coupling structures 121 and one support coupling structure 122, and the second spacing adjustment member 13a may have two side coupling structures 131a and one support coupling structure 132a, but the present invention is not limited thereto. Quantities and sizes of the first base coupling structures 121, and the first support coupling structures 122 of the first spacing adjustment member 12 and the side coupling structure 131a and the center coupling structure 132a of the second spacing adjustment member 13a may be configured according to actual needs.

In this embodiment, the difference between the first relative distance d1 and the second relative distance d2 ranges from 0.3 cm to 3 cm, and ratchets with different tooth heights and ratchet bases with different tooth groove depths may be selected according to actual needs to serve as the first spacing adjustment member 12 and the second spacing adjustment member 13a, such that the difference between the first relative distance d1 and the second relative distance d2 may be adjusted within an appropriate range. Furthermore, given that the first spacing adjustment member 12 has the multiple first base coupling structures 121 and the second spacing adjustment member 13a has the multiple side coupling structures 131a, as well as the first spacing adjustment member 12 having the first support coupling structures 122 with tooth heights greater than those of the first base coupling structures 121, any appropriately sized or type of ratchet and/or ratchet base may be used as the first spacing adjustment member 12. For example, ratchets with suitable module, diameter, number of teeth, tooth width, tooth height, material and/or ratchet base with a suitable tooth groove width, tooth groove depth, the number of tooth grooves, material, can be selected as the first spacing adjustment member 12, and the present invention is not limited thereto.

Additionally, although not illustrated in the drawings for simplicity and to highlight the technical features of this invention, other embodiments may include additional connecting components between the first bracket and the second bracket to provide more stable support. Furthermore, the first spacing adjustment member and second spacing adjustment member may be equipped with protective structures, such as protective casings, to reduce contact with the external environment, which helps to minimize occurrences of issues such as malfunctions and damage.

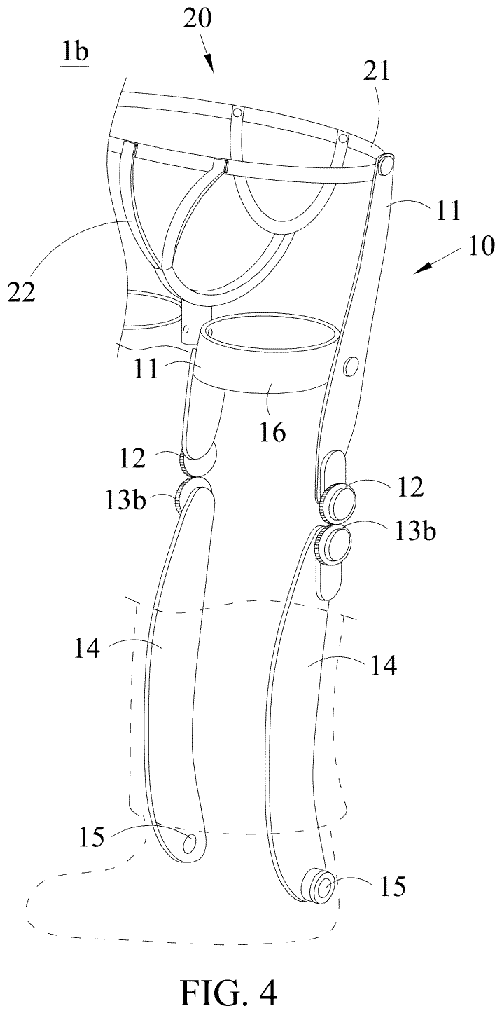

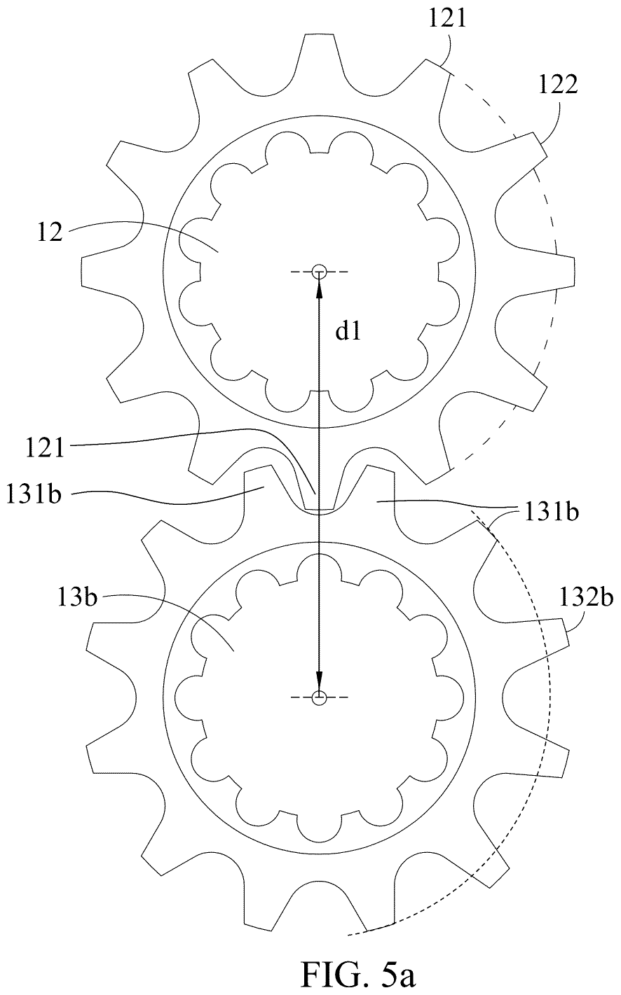

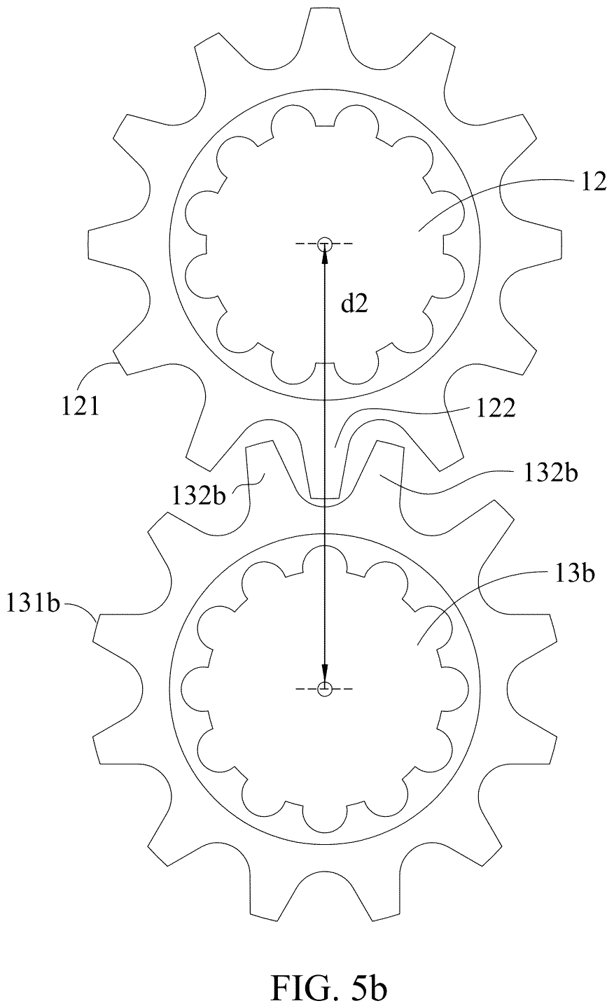

Referring to FIGS. 3, 4, 5a and 5b, FIG. 3 is a schematic diagram of a assist device for a knee joint according to a second embodiment of the present invention; FIG. 4 is a schematic diagram of a assist device for a knee joint according to a third embodiment of the present invention; FIG. 5a is a schematic diagram of a first spacing adjustment member and a second spacing adjustment member of the assist device when the user's knee joint is in a bent state according to the second embodiment and the third embodiment of the present invention; and FIG. 5b is a schematic diagram of the first spacing adjustment member and the second spacing adjustment member of the assist device when the user's knee joint is in an extended state according to the second embodiment and the third embodiment of the present invention.

As shown in FIG. 3, according to the second embodiment of the present invention, an assist device 1b for the knee joint is provided, and the assist device 1b includes at least one knee joint auxiliary mechanism 10, which is arranged at least on a side of a lower limb of a user, and the knee joint assist mechanism each includes a first bracket 11, a first spacing adjustment member 12, a second spacing adjustment member 13b, and a second bracket 14. One end of the first bracket 11 corresponding to an outer side of the thigh is connected to a waist fixation mechanism 20 and pivotally connected to a waist fixing belt 21 of the waist fixing mechanism 20, and one end of the first bracket 11 corresponding to an inner side of the thigh is slidably set on the hip support 22, thereby fixed to the lower limb. The first spacing adjustment member 12 is pivotally connected to another end of the first bracket 11 corresponding to a position of the knee joint of the lower limb, and the second spacing adjustment member 13b is movably engaged with the first spacing adjustment member 12. One end of the second bracket 14 is pivotally connected to the second spacing adjustment member 13b at a position corresponding to the knee joint, and another end of the second bracket 14 contacts the ground.

As shown in FIG. 4, the main difference between this embodiment and the aforementioned embodiment is that, one end of the first bracket 11 corresponding to the outer side of the thigh is connected to the waist fixation mechanism 20, and is fixed to the lower limb through the waist fixation belt 21 pivotally connected to the waist fixation mechanism 20; in addition, the hip support 22 may be provided with a cylindrical member extending downward, and one end of the first bracket 11 corresponding to the inner side of the thigh can be pivotally connected to the cylindrical member and fixed to the lower limb.

In this embodiment, the first spacing adjustment member 12 may have a plurality of first base coupling structures 121 and a plurality of first support coupling structures 122 having sizes greater than that of the plurality of first base coupling structures 121, and the second spacing adjustment member 13b may have a plurality of second base coupling structures 131b and a plurality of second support coupling structures 132b having sizes greater than that of the plurality of second base coupling structures 131b. When the knee joint is in a bent state, the first base coupling structures 121 of the first spacing adjustment member 12 and the second base coupling structures 131b of the second spacing adjustment member 13b are engaged with each other, and a first relative distance d1 is formed between the first spacing adjustment member 12 and the second spacing adjustment member 13b. When the knee joint is in an extended state, the first support coupling structures 122 of the first spacing adjustment member 12 and the second support coupling structures 132b of the second spacing adjustment member 13b are engaged with each other, and a second relative distance d2 is formed between the first spacing adjustment member 12 and the second spacing adjustment member 13b, with the second relative distance d2 being greater than the first relative distance d1.

Specifically, as shown in FIG. 5a, in this embodiment, the first spacing adjustment member 12 and the second spacing adjustment member 13a are both ratchets, and the first spacing adjustment member 12 and the second spacing adjustment member 13b respectively have base ratchet teeth as the first base coupling structures 121 and supporting ratchet teeth as the first support coupling structures 122, and the supporting ratchet teeth have a tooth height greater than that of the base ratchet teeth.

Furthermore, as shown in FIG. 5a, when the knee joint is in a bent state, the base ratchet teeth of the first spacing adjustment member 12 and the base ratchet teeth of the second spacing adjustment member 13b are engaged with one other, and the first relative distance d1 is formed between the first spacing adjustment member 12 and the second spacing adjustment member 13b. Moreover, as shown in FIG. 5b, when the knee joint is in the extended state, the supporting ratchet teeth of the first spacing adjustment member 12 and the supporting ratchet teeth of the second spacing adjustment member 13b are engaged with each other, the second relative distance d2 is formed between the first spacing adjustment member 12 and the second spacing adjustment member 13b, and since the tooth height of the supporting ratchet teeth (i.e., the first support coupling structures 122, and the second support coupling structures 132b) is greater than the tooth height of the base ratchet teeth (i.e., the first base coupling structures 121, and the second base coupling structures 131b), the second relative distance d2 is greater than the first relative distance d1.

Specifically, users suffering from knee joint conditions such as osteoarthritis may wear the assist device 1b of the present invention to provide support for their knee joints. When the user is in a seated position with the knee joint bent, the multiple first base coupling structures 121 of the first spacing adjustment member 12 and the second base coupling structures 131b of the second spacing adjustment member 13b engage with one another, and the first relative distance d1 is formed between the first spacing adjustment member 12 and the second spacing adjustment member 13b. As the user transitions from the seated position with the knee joint bent to a standing position with the knee joint extended, the user's thigh moves the first bracket 11, causing the first spacing adjustment member 12 and the second spacing adjustment member 13b to pivot at their engagement point. The first bracket 11 rotates the first spacing adjustment member 12 along a rotational axis, and the second spacing adjustment member 13b, which is engaged with the first spacing adjustment member 12, follows this movement. This causes the first spacing adjustment member 12 and the second spacing adjustment member 13b to rotate, such that first support coupling structures 122 engage with and the second support coupling structures 132b. Consequently, the second relative distance d2 formed between the first spacing adjustment member 12 and the second spacing adjustment member 13b is larger than the first relative distance d1.

With this configuration, as the user's knee joint transitions from the bent state to the extended state, the engagement between the first spacing adjustment member 12 and the second spacing adjustment member 13b changes in response to the user's movements. This causes the first support coupling structures 122 of the first spacing adjustment member 12 and the second support coupling structures 132b of the second spacing adjustment member 13b to engage with each other. Consequently, the overall height of the knee joint auxiliary mechanism 10 is adjusted through the first support coupling structures 122 and the second support coupling structures 132b, providing support to reduce the pressure on the knee joint, prevent direct friction between the knee joints, and alleviate pain, swelling, and cartilage wear.

Additionally, in this embodiment, the assist device 1b of this invention similarly includes two knee joint auxiliary mechanisms 10, each including two first brackets 11, two first spacing adjustment members 12, two second spacing adjustment members 13a, and two second brackets 14. The two first brackets 11, the two first spacing adjustment members 12, the two second spacing adjustment members 13a, and the two second brackets 14 of each of the knee joint auxiliary mechanisms 10 are positioned on inner and outer sides of one of the user's lower limbs. The specific configuration of the above components is similar to that described in the first embodiment and will not be repeated here.

Furthermore, for the sake of simplicity in the illustration, only the knee joint auxiliary mechanism 10 on the user's left lower limb is shown in FIGS. 3 and 4, while the knee joint auxiliary mechanism 10 on the user's right lower limb is omitted. It is understood by those skilled in the art that the knee joint auxiliary mechanism 10 on the user's left lower limb can have a structure similar to that shown in FIGS. 3 and 4. Therefore, further elaboration is omitted.

It is worth mentioning that in the present embodiment, the waist fixation mechanism 20 includes a waist fixing belt 21 mounted on the waist and a hip support 22 having two ends connected to the waist fixation belt 21 and supporting the hip; in the present embodiment, the two ends of the hip support 22 are generally in a Y-shaped structure. This configuration provides ample support to the hips while avoiding the user's private areas. For example, the Y-shaped part can support or secure the user's buttocks. When the assist device 1b operates in response to the user's movements, the first bracket 11 will cause the waist fixation mechanism 20 to shift, pulling the waist fixation belt 21 and the hip support 22, lifting the user's waist and hips, effectively distributing the user's body weight through the waist fixation belt 21 and the hip support 22 to the two knee joint auxiliary mechanisms 10. Consequently, this reduces the pressure on the knee joints, enhancing the comfort of using the assist device 1b.

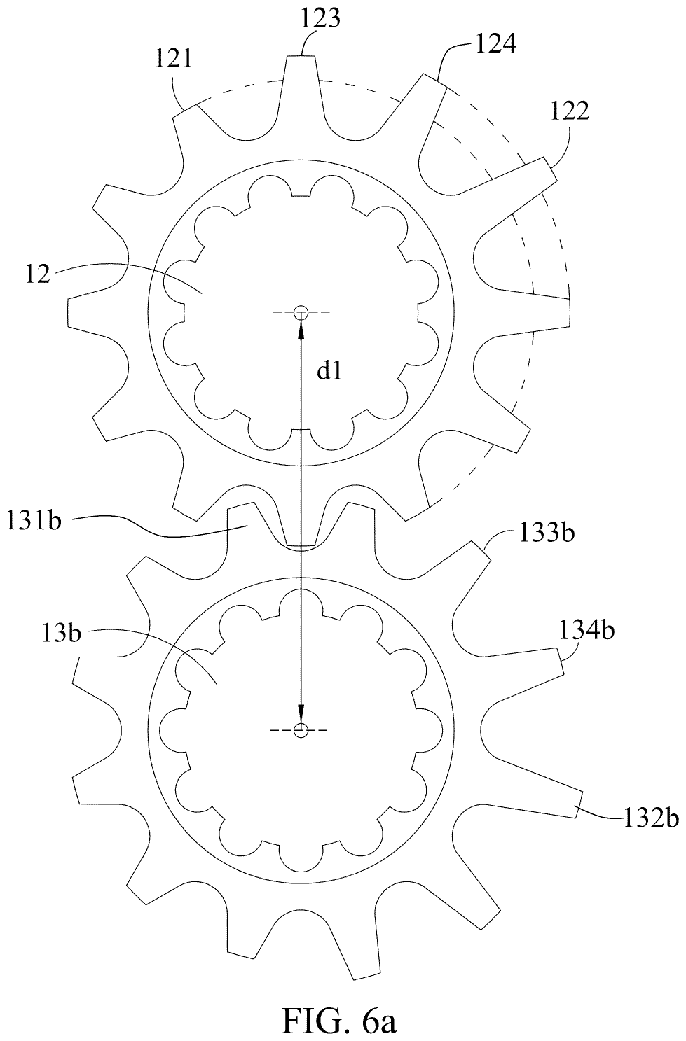

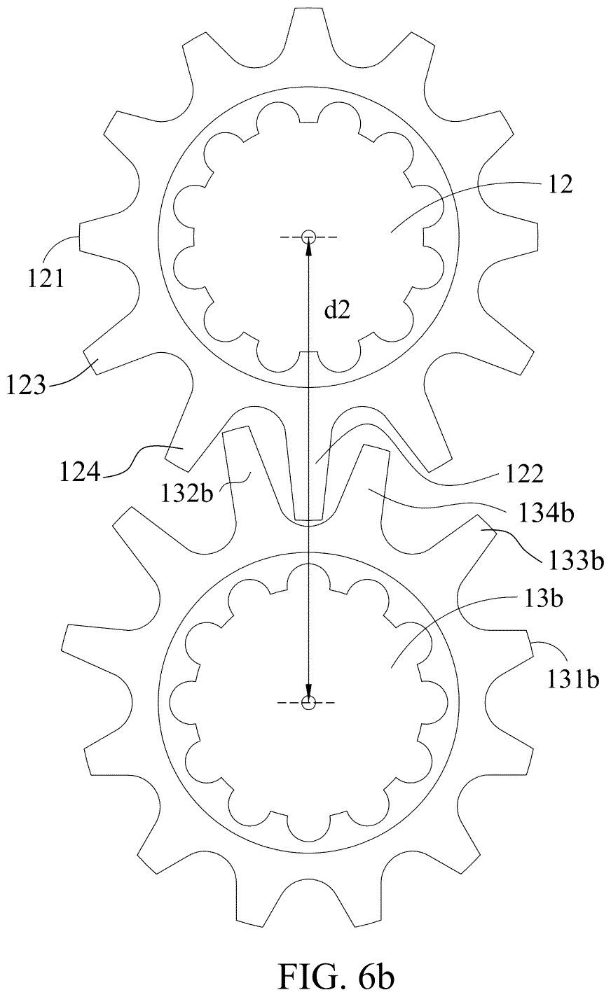

Reference is made to FIGS. 6a and 6b in conjunction with FIG. 1. FIG. 6a is a schematic diagram of a first spacing adjustment member and a second spacing adjustment member of the assist device when the user's knee joint is in a bent state according to the third embodiment of the present invention; and FIG. 6b is a schematic diagram of the first spacing adjustment member and the second spacing adjustment member of the assist device when the user's knee joint is in an extended state according to the third embodiment of the present invention.

In this embodiment, the overall structure thereof is mainly different from the aforementioned embodiment in that, a difference between the second relative distance d2 and the first relative distance d1 gradually changes between the first spacing adjustment member 12 and the second spacing adjustment member 13b. For example, in the first spacing adjustment member 12, one or more intermediate coupling structures 123, 124 may be provided between the base coupling structure 121 and the support coupling structure 122, and the tooth heights of the intermediate coupling structures 123, 124 may gradually increase from the base coupling structure 121 to the support coupling structure 122. Correspondingly, in the second spacing adjustment member 13b, one or more intermediate coupling structures 133b, 134b may be provided between the base coupling structure 131b and the support coupling structure 132b, and the tooth heights of the intermediate coupling structures 133b, 134b may gradually increase from the base coupling structure 131b to the support coupling structure 132b. It is worth mentioning that the maximum value of the difference between the second relative distance d2 and the first relative distance d1 may correspond to the user's greatest pain point. For example, the first spacing adjustment member 12 may be a ratchet, in which three quarters of a circular portion may be set as the first base coupling structures 121, and the remaining quarter may be set as the intermediate coupling structures 123, 124 and the first support coupling structures 122, and the first support coupling structures 122 with the highest tooth height are set at a midpoint of the remaining quarter. Specifically, according to the different maximum pain points of different users, the maximum value of the difference between the second relative distance d2 and the first relative distance d1 (i.e., a position of the highest tooth height of the support coupling structure 122) may be customized and placed, and the tooth height can be set. However, this is just an example, and the present disclosure is limited thereto. In some embodiments, one of the first spacing adjustment member 12 and the second spacing adjustment member 13b may have coupling structures with identical tooth heights, and the other one may have coupling structures with variable tooth heights, such that the relative distance between the first spacing adjustment member 12 and the second spacing adjustment member 13b is adjusted based solely on the other one from among the first spacing adjustment member 12 and the second spacing adjustment member 13b with coupling structures of variable tooth heights.

In other words, as the user's knee joint transitions from the bent state to the extended state, the engagement between the first spacing adjustment member 12 and the second spacing adjustment member 13b changes in response to the user's movements, which gradually alters the overall height of the knee joint auxiliary mechanism 10, providing support in a more gradual manner to reduce the pressure on the knee joint and the direct friction between the knee joints, thereby alleviating pain, swelling, and cartilage wear.

In conclusion, the assist device for the knee joint provided by the present invention features a simple structure and is easy to operate. The assist device can respond to the user's movements to rotate the first spacing adjustment member and the second spacing adjustment member without power components such as motors or power supplies, thereby altering the overall height of the assist device for the knee joint. Both the first spacing adjustment member and the second spacing adjustment member may be equipped with multiple base coupling structures and multiple support coupling structures that are larger than the base coupling structures. For example, the first spacing adjustment member and the second spacing adjustment member can be ratchets with teeth of different sizes. As the user transitions from a sitting position to a standing position, the knee joint moves from a bent state to an extended state, causing the first spacing adjustment member and the second spacing adjustment member to rotate simultaneously, which allows the first spacing adjustment member and the second spacing adjustment member to smoothly transition from a state where the base coupling structures are engaged with one another to a state where the support coupling structures are engaged with one another. Consequently, a center distance between the first spacing adjustment member and the second spacing adjustment member increases, distributing the pressure on the knee joint through the first bracket and the second bracket of the knee joint auxiliary mechanism, as well as the waist fixation mechanism, to the waist, legs, and fect, thereby providing effective knee joint support. The assist device for the knee joint of the present invention can adjust its overall height in response to the user's posture changes. It does not require external power, providing support to the knee joint during posture transitions, so as to effectively prevent direct friction between the knee joints, reducing pain and cartilage wear.

Additionally, the waist fixation mechanism of the assist device for the knee joint provided by the present invention includes a waist fixation belt and a hip support. One end of the hip support is detachably connected to the waist fixation belt through a hip support fixing member, so as to allow users to easily detach or adjust the position of the hip support by unfastening the hip support fixing member, facilitating restroom use without needing to remove the entire assist device for the knee joint. Moreover, the first bracket, the first spacing adjustment member, the second spacing adjustment member, the second bracket, the foot fixation member, and the leg fixation member of each of the knee joint auxiliary mechanism of the assist device for the knee joint of the present invention can be concealed under the user's clothing and shoes, thereby maintaining an aesthetically pleasing appearance while providing effective support to the knee joint.

The present invention has been described with reference to exemplary embodiments. It is understood by those skilled in the art that various changes and equivalent arrangements may be made to the present invention in form and detail without departing from the concept and scope of the present invention as defined in the scope of the patent application. Therefore, the scope of protection of the present invention shall be defined in principle as defined in the scope of the patent application attached hereto.

What is claimed is:

1. An assist device for a knee joint, the assist device comprising: at least one knee joint auxiliary mechanism arranged at least on a side of a lower limb of a user, wherein the knee joint assist mechanism includes:
a first bracket, one end of the first bracket being connected to a waist fixation mechanism and fixed to the lower limb through a waist fixation belt of the waist fixation mechanism;
a first spacing adjustment member pivotally connected to another end of the first bracket corresponding to a position of a knee joint of the lower limb;
a second spacing adjustment member movably engaged with the first spacing adjustment member; and
a second bracket, one end of the second bracket being pivotally being connected to the second spacing adjustment member at a position corresponding to the knee joint, and one other end of the second bracket contacting a ground;
wherein one of the first spacing adjustment member and the second spacing adjustment member is provided with a plurality of base coupling structures and one or more support coupling structures having sizes that are larger than sizes of the plurality of base coupling structures;
wherein, when the knee joint is in a bent state, the one of the first spacing adjustment member the second distance adjusting member is engaged with an other one of the first spacing adjustment member and the second spacing adjustment member by the basic coupling structures, and a first relative distance is formed between the first distance adjusting member and the second distance adjusting member;
wherein, when the knee joint is in an extended state, the one of the first spacing adjustment member and the second spacing adjustment member is engaged with the other one of the first spacing adjustment member and the second spacing adjustment member by the one or more support coupling structures, and a second relative distance is formed between the first spacing adjustment member and the second spacing adjustment member, with the second relative distance being greater than the first relative distance.

2. The assist device according to claim 1, wherein the waist fixation mechanism further includes a hip support having one end connected to the waist fixation belt and supporting a hip;
wherein the waist fixation belt is sleeved onto a lower side of the user's anterior superior iliac spine;
wherein the hip support is a plate-shaped structure corresponding to a shape of the hip, and one end of the hip support is detachably connected to the waist fixation belt through a hip support fixing member.

3. The assist device according to claim 1, wherein the waist fixing mechanism is worn on the lower limb and pants-shaped, and the waist fixation mechanism includes the waist fixation belt sleeved onto the waist, two thigh fixation belts respectively sleeved onto two upper thighs, and two thigh lifting belts connected between the waist fixation belt and the two thigh fixation belts;
wherein, the first bracket of the at least one knee joint auxiliary mechanism is fixed to the lower limb through the waist fixation mechanism.

4. The assist device according to claim 1, wherein a difference between the second relative distance and the first relative distance is caused to gradually increases by one or more intermediate coupling structures between the base coupling structures and the one or more support coupling structures, with heights of the one or more intermediate coupling structures gradually increase from a direction from the base coupling structures to the one or more support coupling structures.

5. The assist device according to claim 4, wherein a maximum value of the difference between the second relative distance and the first relative distance is configured to be corresponding to a maximum pain point of the user.

6. The assist device according to claim 5, comprising two of the knee joint auxiliary mechanism, and each of the two knee joint assist mechanisms includes the first bracket, the first spacing adjustment member, the second spacing adjustment member and the second bracket that are arranged on an outer side of the lower limb, and each of the first bracket arranged on the outer side of the lower limb is fixed to the lower limb through the waist fixation mechanism.

7. The assist device according to claim 6, wherein each of the two knee joint assist mechanisms includes another first bracket, another first spacing adjustment member, another second spacing adjustment member, and another second bracket that are arranged on an inner side of the lower limb, and the first brackets respectively arranged on the outer side and the inner side of the lower limb are both fixed to the lower limb through the waist fixation mechanism.

8. The assist device according to claim 5, wherein the first spacing adjustment member is a ratchet, and the second spacing adjustment member is a ratchet or a ratchet base; wherein the plurality of base coupling structures and the one or more support coupling structures of the first spacing adjustment member are ratchet teeth, and the second spacing adjustment member are provided with ratchet teeth or tooth grooves corresponding to the plurality of base coupling structures and the one or more support coupling structures of the first spacing adjustment member.

9. The assist device according to claim 5, wherein the knee joint auxiliary mechanism includes at least one leg fixation member in a ring shape surrounding the user's leg.

10. The assist device according to claim 5, wherein the knee joint assist mechanism includes at least one foot fixation member, which is arranged at the other end of the second bracket and is detachably connected to the second bracket and a side surface of the user's shoe to fix the other end of the second bracket to the user's foot.
